(19) United States
(12) Patent Application Publication
(10) Pub. No.: US 2006/0171971 A1
(43) Pub. Date: Aug. 3, 2006

(54) Composition for wet wipes containing a non-irritating skin health benefit ingredient and the process for making

(75) Inventors: Randall Glenn Marsh, West Chester, OH (US); Philip Andrew Sawin, Liberty Township, OH (US); Victor Nicholas Vega, Cincinnati, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 11/048,446

(22) Filed: Feb. 1, 2005

(51) Int. Cl. A61K 8/19

(52) U.S. Cl. 424/401; 424/70.1

(57) ABSTRACT

An oil-in-water emulsion composition for wet-wipes delivering a particulate skin health benefit ingredient in the oil phase. A process for making an oil-in-water emulsion composition in which the oil phase is first made as a concentrate and then diluted in water.

### FIELD OF THE INVENTION

This invention relates to a composition for a cleansing wipe delivering an improved cleansing performance while simultaneously providing healing and protective benefits to the user. The invention relates to the use of a composition comprising an emollient, a surfactant and/or emulsifier, a rheology modifier, and one or more skin health benefit ingredients, selected to deliver the intended skin health benefits.

The present invention relates to personal care compositions, more particularly personal cleansing and/or skin treating compositions also providing healing and protective benefits. The composition of the present invention may be useful, for example, for gentle perineal and/or peri-anal cleansing and for protection against perineal dermatoses.

### BACKGROUND OF THE INVENTION

The skin is a natural barrier to the penetration of many foreign substances. At times, the natural ability of the skin to protect itself may be compromised by external factors including abrasions, irritants, and the like. Attempts have been made to promote skin health through the use of various products containing additives, or by developing synthetic or naturally occurring polymers that mimic or complement the properties of skin in order to maintain skin health.

Enhancing skin health and delivering agents to the skin to promote skin health has many advantages including: 1) protecting the skin and maintaining the skin in a pristine state, essentially free from chapping or irritation; 2) pH buffering and barrier enhancement to maintain or enhance such base properties of skin; 3) inhibition of irritants that are suspected to promote irritant or allergic contact dermatitis; and 4) maintaining the lubricity of skin.

Cleansing the skin may be a personal hygiene problem not always easily solved. Dry tissue products are the most commonly used cleaning products post-defecation or post-urine release. Dry tissue products are usually referred to as “toilet tissue” or “toilet paper.” Beside the use of dry tissue, wet wipes are frequently used for the purpose of cleaning the anus, perinea, and the peri-anal body area after defecation. So called “wet wipes” are a fibrous structure impregnated with a composition, usually water or oil-based.

For the purpose of the present document, the anus, the peri-anal area, the perinea and the perineal area are all terms indicating the body area of the pelvis between, around and including the anus and the external genitalia. Those terms are used interchangeably and with the same meaning.

The peri-anal skin is marked by the presence of fine folds and wrinkles (sulci) and by hair follicles, both of which make the perineal region one of the more difficult anatomical areas to cleanse. During defecation, fecal matter is excreted through the anus and tends to accumulate in hard to reach locations such as around the base of hairs and in the sulci of the skin's surface.

As the fecal matter dehydrates upon exposure to the air or upon contact with an absorbent cleansing implement such as tissue paper, it adheres more tenaciously to the skin and hair, thus making subsequent removal of the remaining dehydrated soil even more difficult. Generally, common hygienic concerns make the benefits of a good perineal cleansing after defecation and after urine release very relevant to baby, children, and adults. Among those negatives associated with the failure of adequate cleansing are irritation, redness, desquamation, infections, unpleasant odor, or other kinds of personal discomfort or health related concerns.

People suffering from pathological conditions (such as hemorrhoids, fissures, cryptitis, etc) are even more susceptible to those issues and discomfort. For them, as for any person, cleansing must be efficient in terms of removal of fecal residues and gentle in terms of absence of irritation caused by the cleansing. Wet wipes bring a response to that basic need. In comparison to dry toilet tissue, wet wipes have several benefits such as: enabling better lubrication during the use of the wipe; reducing the abrasiveness of the cleansing operation; softening of the residues, enhancing their removal from the skin; softening of the skin tissue; and the ability to deliver a lotion to the skin that can remain on the skin after the cleansing operation.

Altogether, achieving an improved level of cleanliness and reducing irritation during and after cleaning and wiping still remain as basic needs to consumers. There is a need for an emulsion composition providing sufficient cleansing benefits to the skin. There is a need for a wipe that does not irritate, erode the user's skin. nor enhance redness. Additionally, there is a need for a wipe that reduces or eliminates the activity of irritants and promotes healing of any pre-existing or new skin irritation and damage. Further, there is a need for a wipe providing a smooth, long lasting comfortable feeling to the user.

Traditional lotion compositions for use on wet-wipes are manufactured by mixing a surfactant or emulsifier into an excess of water with or without an emollient compound. This process, however, requires stringent process conditions such as high energy input to homogenize the oil and water phase and to generate a defined particle size distribution in the aqueous phase. Additionally, high shear mixing will generally be needed together with heating of the composition being prepared. There is a need for a process to prepare such wipes comprising an emulsion that would preferably only require low energy input, and in particular low shear and little or no heat input. There is also a need for a process to prepare such wipes comprising an emulsion that would provide a low shear stress to the ingredients. There is additionally a need for a way to prepare an emulsion exhibiting oil droplets that are of small dimension and are stable and/or meta-stable. There is a need for an emulsion composition applied to a wipe that is relatively easy and cost efficient to prepare and preserve. There is additionally a need for a way to prepare such emulsions that would enable storage and transportation of the emulsion in a concentrated form.

There is a need for the preparation of a universal emulsion premix or concentrate to which the various additives corresponding to the desired properties could be added. There is a need for a way to prepare such an emulsion combining all or most of the above benefits to a so far unachieved level.

There is also the need for a wipe that has a long shelf life and whose composition reduces the proliferation of microorganisms in the package and during use. Finally there is a need for a wipe combining all or most of the above benefits to a so far unachieved level.

### SUMMARY OF THE INVENTION

The present invention describes an oil-in-water emulsion composition that can be used for a body-cleaning wipe intended to clean areas such as the perineum after defecation or the release of urine. The present invention addresses the problem of delivering a skin health benefit ingredient in the oil phase of the composition.

In one embodiment, the composition includes an oil phase comprising an emollient, an emulsifier, and a particulate skin health benefit ingredient. The water phase comprises a rheology modifier. In yet another embodiment, the composition may further comprise preservative and/or other adjunct ingredients.

In another embodiment of the invention, the composition includes an oil phase comprising an emollient, an emulsifier, and a particulate skin health benefit ingredient and a water phase which comprises a rheology modifier wherein the overall pH of the composition is from about 6 to about 9.

In another embodiment the composition reduces the IL-1α response to irritating bowel movement by at least about 40% while maintaining skin cell viability of greater than about 70%.

In another aspect, the present invention comprises a wet-wipe comprising such compositions as noted above.

In another aspect, the present invention comprises a wet-wipe comprising such a composition as noted above wherein the particulate skin health ingredient has a solubility of less than about 1% in water at 25° C.

In another aspect, the present invention comprises a wet-wipe comprising such a composition as noted above wherein the particulate skin health ingredient is a zinc compound.

The present invention describes a process for making a wet-wipe comprising an oil-in-water emulsion composition as noted above. The present invention describes a process for preparing the lotion composition from a concentrated emulsion that can be easily handled, stored, and transported. The concentrated emulsion composition can then be diluted with water and complemented with other compounds to obtain the desired final cleansing composition that may be applied to a wipe substrate.

The composition concentrate of the present invention, when diluted with water, may lead to a small oil droplet size.

### DETAILED DESCRIPTION OF THE INVENTION

Wet-Wipes:

The present invention provides a composition for wet-wipes. “Wet-wipes” is the general term to describe a piece of material, generally non-woven material, used to cleanse body parts. In particular, many currently available wipes may be intended for the cleansing of the peri-anal area after defecation. Other wipes may be available for the cleansing of the face or other body parts. The present invention focuses on, but is not limited to, wipes for the peri-anal (or perineal) area.

Wet-wipes substrates may be generally of sufficient dimension to allow for convenient handling. Typically, the substrate may be cut and/or folded to such dimensions as part of the manufacturing process. In some instances, the substrate may be cut into individual portions so as to provide separate wipes which are often stacked and interleaved in consumer packaging. In other embodiments, the wipes are in a web form where the web has been slit and folded to a predetermined width and provided with means (e.g., perforations) to allow individual wipes to be separated from the web by a user. Suitably, an individual wet wipe according to the present invention may have a length between about 100 mm and about 250 mm and a width between about 140 mm and about 250 mm. In one embodiment, the wipe may be about 200 mm long and about 180 mm wide.

The material of the wipe may generally be soft and flexible, potentially having a structured surface to enhance its cleaning performance. The material may be a non-woven material, generally made of synthetic compounds. However, woven materials, as well as the use of natural compounds in either woven or non-woven materials are within the scope of the present invention. In one embodiment of the present invention, the non-woven material comprises fibers selected from the group consisting of polyolefin, polyester, cellulose (including rayon and lyocell), polyamides, polyesteramide, polyvinyl alcohols, and combinations thereof. The substrate usable for this invention can be manufactured via any suitable process, including air laying, wet laying, carding, spun bonding, and melt blowing and combinations of such production processes. The substrate materials may also be treated to improve the softness and texture thereof by processes such as hydroentanglement or spunlacing. The substrate may have a basis weight of at least about 40 grams/M². In one embodiment, the substrate may have a basis weight of at least about 45 grams/M². In another embodiment, the substrate basis weight may be less than about 100 grams/m². In another embodiment, substrates may have a basis weight between about 45 grams/M² and about 75 grams/M², and in yet another embodiment a basis weight between about 45 grams/m² and about 65 grams/m².

Substrate materials may comprise a blend of hydrophilic fibers or hydrophilically modified fibers to insure the substrate is able to imbibe a suitable amount of the cleansing composition and stiffening fibers to provide a desirable “hand” to the substrate. The substrate may also comprise a layered structure so as to put soft, flexible fibers on the outer surfaces thereof and to “hide” the stiffening fibers in the interior.

A suitable substrate may be a carded nonwoven comprising a 40/60 blend of viscose fibers and polypropylene fibers having a basis weight of 58 grams/M² as available from Suominen of Tampere, Finland as FIBRELLA 3160. Another suitable material for use as a substrate may be SAWATEX 2642 as available from Sandler AG of Schwarzenbach/Salle, Germany. Yet another suitable material for use as a substrate may have a basis weight of from about 50 grams/M² to about 60 grams/M² and have a 20/80 blend of viscose fibers and polypropylene fibers.

Cleansing Composition

Wet-wipes may be impregnated with a liquid or semi-liquid composition, intended to both facilitate cleaning and to deposit a skin benefit agent onto the skin. Other ingredients or actives (for example cosmetic actives) may be part of the composition.

Generally the composition may be of sufficiently low viscosity to disperse solid soils disposed on the skin and to facilitate wetting of the entire structure of the wipe. In some other instances, the composition can be primarily present at the wipe surface and to a lesser extent in the inner structure of the wipe. In one embodiment, the substrate may be impregnated with at least about 2 times its weight with the cleaning lotion. In another embodiment, the wipe may be impregnated with at least about 2.5 times its weight, and in yet another embodiment, with at least about 3 times its weight. In a further embodiment, the substrate may be impregnated with less than about 5 times its weight.

Desirably, the substrate releasably carries the cleansing composition. Therefore, the cleansing composition may be contained either in or on the substrate and can be readily released from the substrate by applying a relatively low force to the substrate (e.g., wiping a surface, such as the skin in the peri-anal area, with the wet wipe).

In most of its embodiments, the cleansing composition of the present invention comprises, but is not limited to: an emollient, an emulsifier, a skin health benefit ingredient, a rheology modifier, and water. Other ingredients may be incorporated into the composition, including, but not limited to, soothing agents, vitamins, minerals, antioxidants, moisturizers, botanicals, perfumes, potentiators, aesthetic enhancing ingredients, a preservative or a multiplicity of preservatives acting together as a preservative system and additional skin health benefit ingredients. It is to be noted that some compounds can have multiple functions and that all compounds are not necessarily present in the composition of the invention. The composition of the present invention may be a so called oil-in-water emulsion in that small oil droplets are dispersed and surrounded by a hydrophilic medium. The composition may be an oil-in-water emulsion composition wherein the skin health benefit ingredient is suspended in the oil phase. Without being limited by theory, it is believed that having the skin health benefit ingredient suspended in the oil phase may provide better and longer lasting skin coverage, less agglomeration of the skin health benefit ingredient and better “wetting” of the skin health benefit ingredient which may result in less settling and increased lotion stability.

The cleansing composition of the present invention may have a water content level of greater than about 60%. In another embodiment the water content level may be greater than about 70% and may be greater than about 85% in yet another embodiment.

The cleansing composition of the present invention may provide a protecting and healing effect as evidenced by a reduced irritation response of skin to infant feces (as assessed by measuring inflammation according to the EPIDERM method described in the TEST METHODS section). Additionally, the cleansing composition itself may be mild and essentially non-irritating (as assessed by measuring skin cell viability according to the EPIDERM method described in the TEST METHODS section). In one embodiment, the cleansing composition may provide at least about 40% mean reduction in the inflammatory marker interleukin-l alpha (hereinafter “IL-1α) while maintaining skin cell viability of greater than about 70%.

The pH of the cleansing composition in one embodiment may be from about 6 to about 9. In another embodiment the pH may be from about 6 to about 8 and in yet another embodiment may be from about 7 to about 8.
Concentrated Emulsion Composition

The composition of the present invention may originate from a concentrated emulsion composition where the concentrated emulsion composition is diluted by an aqueous medium. The concentrated emulsion composition of the present invention may have a water content of about 0% to about 30%. In one embodiment, the water content may be less than about 30% (w/w) (defined as the weight of the water in the concentrate over the weight of all ingredients in the concentrate×100), in another embodiment the water content may be less than about 20% (w/w), and in yet another embodiment the water content may be less than about 10% (w/w). The minimum amount of water may be 0%. The concentrated emulsion concentrate may also comprise an emollient, an emulsifier, and a particulate skin health benefit ingredient.

Emollient

The concentrated emulsion composition contains an emollient. Common dictionaries define “emollient” as “something that softens or soothes.” The functions of emollients in a wet-wipe include: (1) improving the glide of the wipe on the skin, by enhancing the lubrication and thus decreasing the abrasion of the skin; (2) softening the residues (e.g., fecal residues or dried urine residues), thus enhancing their removal from the skin; (3) softening the skin, thus reducing its dryness while improving its flexibility under the wiping movement, and; (4) protecting the skin as the emollient is deposited onto the skin and remains at its surface as a thin protective layer.

In one embodiment of the present invention the average emollient droplet size may be less than about 12 microns, in another embodiment less than about 10 microns, in yet another embodiment less than about 5 microns, and in yet another embodiment still less than about 1 micron. To this corresponds an average particle size of the diluted composition having the same size. Some variation in the particle size can be observed during/after the dilution step.

The emollient of the present invention may have a solubility parameter between about 5 and about 12, and in another embodiment between about 5 and about 9. The solubility parameter is defined by Hildebrand as the sum of all the cohesive forces and the square root of the energy of vaporization: δ=(ΔEv/V)^{1/2} where V =molecular weight/density and Ev=heat of vaporization. (This is from C.D. Vaughan, J Soc Cosmet Chem, Vol. 36, September/October, 1985). The amount of emollient that can be included in the lotion composition will depend on a variety of factors, including the particular emollient involved, the lotion-like benefits desired, and the other components of the lotion composition. It has been found that compositions with low or very low emollient content may be best suited for the invention. The emollient content of the composition may be from about 0.001% to less than about 5%, in another embodiment from 0.001% to less than about 3%, and in yet another embodiment from about 0.001% to less than about 2.5% (all % are weight/weight % of the emollient in the composition).

Some emollients that may be used in the present invention are silicon based. Silicone based emollients are organo-silicone based polymers with repeating siloxane (Si-O) units. Silicone based emollients of the present invention are hydrophobic and exist in a wide range of possible molecular weights. They include linear, cyclic and cross-linked varieties. Silicone oils are generally chemically inert and usually have a high flash point. Due to their low surface tension, silicone oils readily spread and have high surface activity. Examples of silicone oils that may be suitable for use in the present invention include, but are not limited to, Cyclomethicones, Dimethicones, phenyl-modified silicones, alkyl-modified silicones, silicone resins, and combinations thereof.

Emollients useful in the present invention may also be unsaturated esters or fatty esters (saturated or unsaturated). Examples of unsaturated esters or fatty esters of the present invention include: Caprylic Capric Triglycerides in combination with Bis-PEG/PPG-16/16 PEG/PPG-16/16 Dimethicone, and C₁₂-C₁₅ Alkylbenzoate.

Emollients particularly suited for the present invention may be selected from a list comprising Dimethiconol, Dimethicone, Cyclopentasiloxane, Caprylic/Capric Triglyceride, C₁₂-C₁₅ Alyklbenzoate, mineral oil, or a mixture of Caprylic Capric Triglyceride and Bis-PEG/PPG-16/16 PEG/PPG-16/16 Dimethicone known as ABIL CARE 85™ (as is available from Degussa Care Specialties of Hopewell, VA), or a mixture of Caprylic Capric Triglyceride and mineral oil, and any combinations thereof.

Emulsifier

The concentrated emulsion composition also includes an emulsifier such as those forming oil-in-water emulsions. As defined, an emulsifier is a surface-active agent that promotes the formation of an emulsion. Included among emulsifiers are, for example, polymeric emulsifiers, conventional emulsifiers, silicone based emulsifiers, and commonly known surfactants. Examples of commonly known surfactants are, for example, sucrose and glucose esters and derivatives, ethoxylated fatty acids, sorbitan derivatives, ethoxylated fatty esters and block copolymers.

Emulsifiers may be nonionic surfactants. Examples of nonionic surfactants are disclosed in McCutcheon's, Detergents and Emulsifiers, North American Edition (1997) and McCutcheon's, Functional Materials, North American Edition (1997) both published by Mc Publishing Co. of Glen Rock, NJ.

The emulsifier may be employed in an amount effective to emulsify the emollient and other non-water-soluble oil phase components that may be present in the composition such as an amount ranging from about 0.001% to about 4%, further from about 0.01% to about 2%, and even further from about 0.02% to about 1% (based on the weight emulsifiers over the weight of the composition). Mixtures of emulsifiers may be used.

Non-limiting examples of emulsifiers may be alkylpolyglucosides, decylpolyglucosides, fatty alcohols, cationic surfactants such as, but not limited to, poly-12 Hydroxy Stearic Acid, quaternary compounds, and protein-based surfactants, or a mixture of Caprylic Capric Triglyceride and Bis-PEG/PPG-16/16 PEG/PPG-16/16 Dimethicone, Polysorbate 20, and combinations thereof.

Skin Health Benefit Ingredient

A third component of the concentrated emulsion composition is a skin health benefit ingredient. The term “skin health benefit ingredient” as used herein refers to materials that, when delivered topically to the skin, are capable of preventing, reducing, and/or limiting occurrences of skin disorders, particularly skin disorders associated with exposure of the skin to urine and/or feces. The term “skin disorders” as used herein refers to symptoms associated with irritating, acute or chronic skin abnormalities. Examples of such symptoms include, but are not limited to, rash, itching, inflammation, burning, stinging, redness, swelling, sensitivity, sensation of heat, flaking/scaling, malodor, and the like.

Skin health benefit ingredients suitable for the present invention may include particulate zinc containing material that has the following properties: (1) low solubility in water (less than 1% at 25° C.), and; (2) provides labile zinc (as determined by the LABILITY method described in the TEST METHODS section) at a pH of 6-9. In one embodiment the particulate zinc material may have a solubility of less than about 0.5% at 25° C., and in another embodiment the particulate zinc material may have a solubility of less than about 0.25% at 25° C.. Using this methodology, examples of particulate zinc materials having available Zn²⁺ include zinc oxide, zinc stearate, zinc carbonate, and zinc monoglycerolate. Examples of particulate zinc containing material include, but are not limited to, those compounds of a conventional particulate size or of a nanoparticle size. Conventional particle sizes may be in the range of a mean particle size of from about 0. 0 micron to about 0.40 micron and may have a surface area from about 3.0 square meters/gram to about 9.5 square meters/gram. Nanoparticle sizes may be in the range of from about 0.2 nm to about 100 nm.

Examples of zinc particulate material useful in certain embodiments of the present invention include the following:

Inorganic Materials: Zinc aluminate, zinc carbonate, zinc oxide and materials containing zinc oxide (i.e., calamine), zinc phosphates (i.e., orthophosphate and pyrophosphate), zinc selenide, zinc sulfide, zinc silicates (i.e., ortho- and meta-zinc silicates), zinc silicofluoride, zinc borate, zinc hydroxide and hydroxy sulfate, zinc-containing layered materials and combinations thereof.

Layered Materials: Layered zinc particulate materials are those with crystal growth primarily occurring in two dimensions. Conventionally, layered structures are not only those in which all of the atoms are incorporated in well-defined layers, but also those in which there are ions or molecules between the layers, called gallery ions (A. F. Wells “Structural Inorganic Chemistry”. Clarendon Press, 1975). Zinc-containing layered materials may have zinc incorporated in the layers and/or as more labile components of the gallery ions.

Many zinc-containing layered materials occur naturally as minerals. Common examples include hydrozincite (zinc carbonate hydroxide), basic zinc carbonate, aurichalcite (zinc copper carbonate hydroxide), rosasite (copper zinc carbonate hydroxide), and many related minerals.

Natural zinc-containing layered materials can also occur wherein anionic layer species such as clay-type minerals (e.g., phyllosilicates) contain ion-exchanged zinc gallery ions.

Another common class of zinc-containing layered materials may be layered double hydroxides, which are generally represented by the formula [M²⁺₁₋ₓM³⁺ₓ(OH)₂]^{x+}A^{m-}_{x/m}·nH2O, where some or all of the divalent ions (M²⁺) would be represented as zinc ions (E. L. Crepaldi et al. (2002), J. Colloid Interfac. Sci. 248:429-442).

Yet another class of zinc-containing layered materials may be hydroxy double compounds (H. Morioka et al. (1999), Inorganic Chemistry 38:4211-4216). Hydroxy double compounds can be represented by the general formula [M²⁺₁₋ₓM²⁺₁₊ₓ(OH)_{3(1-y)]+}Aⁿ⁻_{(1-3y)/n}·nH2O where the two metal ions may be different. If the two metal ions are the same and represented by zinc, the formula simplifies to [Zn₁₊ₓ(OH)₂]^{2x+}2x A⁻·nH₂O. This latter formula represents common materials such as zinc hydroxychloride and zinc hydroxynitrate. These are related to hydrozincite, wherein the divalent anion is replaced by a monovalent anion.

These classes of zinc-containing layered materials represent relatively common examples of the general category and are not intended to be limiting as to the broader scope of materials which fit this definition.

Natural Zinc-Containing Ores and Minerals: Sphalerite (zinc blend), Wurtzite, Smithsonite, Franklinite, Zincite, Willemite, Troostite, Hemimorphite, and combinations thereof.

Organic Materials: Zinc fatty acids (i.e., caproate, laurate, oleate, stearate, undecylate, etc.), zinc alkyl sulfonic acids, zinc naphthenate, zinc tartrate, zinc tannate, zinc monoglycerolate, zinc allantoinate, zinc urate, zinc amino acids (i.e., methionate, phenylalinate, tryptophanate, cysteinate, etc.) and combinations thereof.

Polymeric Materials: Zinc polycarboxylates (i.e., polyacrylate), zinc polysulfate, and combinations thereof.

Physically-Adsorbed Forms: Zinc-loaded ion exchange resins, zinc adsorbed on particle or microbead surfaces, composite particles in which zinc materials are incorporated, (i.e., as core/shell or aggregate morphologies), and combinations thereof.

Zinc particulate materials useful for the present invention include, but are not limited to zinc oxide, zinc carbonate, zinc stearate, zinc monoglycerolate, and zinc-containing layer materials. These materials can be natural, obtained synthetically, or formed in situ in a composition or during a production process.

Commercially available sources of zinc oxide include Z-Cote™ and Z-Cote HPI™ (BASF, Florham Park, New Jersey), USP-1 zinc oxide and USP-2 zinc oxide (available from Zinc Corporation of America, Monaca, Pa.), and ULTRAFINE ₃₅₀™ zinc oxide (available from Kobo Products, Inc., South Plainfield, N.J.). Commercially available sources of zinc oxide dispersions include: premixes of zinc oxide and a dispersing agent such as polyhydroxystearic acid in oils (available from Uniqema Inc., Wilmington, Del.) under the tradename Spectraveil™, including a premix of zinc oxide and dispersing agent in caprylic/capric triglycerides and mineral oil under the tradename Spectraveil MOTG™; and a premix of zinc oxide and a dispersing agent such as polyglyceryl-6 polyricinoleate (available from Kobo Products Inc., South Plainfield, N.J. under the class of “High Solids® Dispersions” or “ZnO Attenuation Grade Dispersions”), including a premix of zinc oxide and dispersing agent in C₁₂-C₁₅ alkyl benzoate under the tradename TNH70MZ8N™.

Commercially available sources of zinc carbonate include Zinc Carbonate Basic (Cater Chemicals: Bensenville, Ill.), Zinc Carbonate (Shepherd Chemicals: Norwood, Ohio), Zinc Carbonate (CPS Union Corp.: New York, N.Y.), Zinc Carbonate (Elementis Pigments: Durham, UK), and Zinc Carbonate AC™ (Bruggemann Chemical: Newtown Square, Pa.).

Without being bound by theory, it is believed that the low water solubility and high lability of the particulate zinc materials enables them to provide a sustained, slow-release of zinc ion to the skin as required, thereby avoiding irritation that may be caused when local levels of zinc ion are high (such as with the use of highly soluble zinc salts).

Skin health benefit ingredients suitable for the present invention may also include gold containing material, silver containing material, platinum containing material and combinations thereof. Examples of gold containing material, silver containing material and platinum containing material include, but are not limited to, the following:

Inorganic Materials: Gold (I) bromide, gold (I) chloride, gold (I) iodide, gold (I) sulfide, gold (III) hydroxide, gold (III) oxide, gold (III) selenate, gold (III) selenide, silver (I) bromate, silver (I) bromide, silver (I) carbonate, silver (I) chloride, silver (I) chlorite, silver (I) iodate, silver (I) iodide, silver (I) metaphosphate, silver (I) nitrite, silver (I) oxalate, silver (I) oxide, silver (I) permanganate, silver (I) phosphate, silver (I) selenate, silver (I) selenide, silver (I) sulfate, silver (I) sulfide, silver (I) sulfite, silver (II) oxalate, silver (II) oxide, platinum (II) bromide, platinum (II) chloride, platinum (II) iodide, platinum (II) oxide, platinum (IV) bromide, platinum (IV) oxide, platinum (I) dichloride, platinum (I) hydroxide, platinum (I) monosulfide, platinum (I) disulfide, platinum (I) sesquisulfide, and combinations thereof.

Natural Gold-Containing Ores and Minerals: The gold Tellurides (including Calaverite, Sylvanite, Kostovite, Krennerite, Nagyagite, Petzite, Muthmannite, and Montbrayite), the gold amalgams (including Auricupride, Aurosmiridium, Aurocupride, Aurostibite, Electrum, Fischeserti, Maldonite, Porpezite, Rhodite, and Uytenbogaardite), and combinations thereof.

Natural Silver-Containing Ores and Minerals: Prousite, Pyrargyrite, Acanthite, Argentite, Cerargyrite, Sylvanite, and combinations thereof.

Natural Platinum-Containing Ores and Minerals: Sperrylite.

Organic Materials: Silver (I) acetate, silver (1) benzoate, silver (I) citrate, and combinations thereof.

Physically-Adsorbed Forms: Gold-loaded ion exchange resins, gold adsorbed on particle or microbead surfaces, composite particles in which gold materials are incorporated, (i.e., as core/shell or aggregate morphologies), Silver-loaded ion exchange resins, silver adsorbed on particle or microbead surfaces, composite particles in which silver materials are incorporated, (i.e., as core/shell or aggregate morphologies), Platinum-loaded ion exchange resins, platinum adsorbed on particle or microbead surfaces, composite particles in which platinum materials are incorporated, (i.e., as core/shell or aggregate morphologies), and combinations thereof.

The skin health benefit ingredient may be present in a range from about 0.001% to at least about 10% by weight of the composition, in another embodiment from about 0.01% to about at least 5%, and in yet another embodiment from about 0.1% to about at least 3%.

Rheology Modifier

It has been found that the rheology of the cleansing composition plays a role in its functionality. Generally the cleansing composition should be of sufficiently low viscosity to disperse solid soils disposed on the skin and to facilitate impregnation of the entire structure of the wipe.

Rheology modifiers are compounds that increase the viscosity of the cleansing composition at lower temperatures as well as at process temperatures. Rheology modifiers also provide “structure” to the cleansing compositions to prevent settling out (separation) of insoluble and partially soluble components.

The effect and advantage of rheology modifiers are in particular described in U.S. Patent Application Ser. No. 20020128621A1 entitled “Absorbent articles with simplified compositions having good stability” published on Sep. 12, 2002, filed on Dec. 21, 2001, by Kruchoski et al., and U.S. Patent Application Ser. No. 20020128615A1 entitled “Absorbent articles with non-aqueous compositions containing anionic polymers” published on Sep. 12, 2002, filed on Dec. 22, 2001, by Tyrrell et al.

In addition to stabilizing the suspension of insoluble and partially soluble components, the rheology modifiers of the invention also help to stabilize the cleansing composition on the wipe and enhance the transfer of lotion to the skin. The wiping movement increases the shear and pressure therefore decreasing the viscosity of the lotion and enabling a better transfer to the skin as well as a better lubrication effect.

Additionally, the rheology modifier helps to preserve a homogeneous distribution of the composition within the wipe stack: Any fluid composition has a tendency to migrate to the lower part of the wipes stack during prolonged storage. This effect creates an upper zone of the stack having less composition than the bottom part. This may be seen as a sign of relatively low quality by the users.

Rheology modifiers useful for the present application may exhibit low initial viscosity and high yield. Low initial viscosity may be less than about 1500 cP, and further may be less than about 1000 cP, and in yet another embodiment may be less than about 500 cP. The yield value or yield stress is the minimum amount of force necessary to induce flow. The yield value may be from about 0 to about 5 Pa. Further the yield value may be from about 0 to about 4 Pa and in yet another embodiment the yield value may be from about 0 to about 3 Pa. Particularly suited for the present invention are rheology modifiers such as, but not limited to:
Blends of material as are available from Uniqema GmbH&Co. KG, of Emmerich, Germany under the trade name ARLATONE™ such as, but not limited to, ARLATONE V-175™ which blends sucrose palmitate, glyceryl stearate, glyceryl stearate citrate, sucrose, mannan, and xanthan gum, and ARLATONE V-100™ which blends Steareth-100, Steareth-2, glyceryl stearate citrate, sucrose, mannan, and xanthan gum.
Blends of materials as are available from Seppic France of Paris, France as SIMULGEL™, such as, but not limited to, SIMULGEL NS™ which comprises a blend of a hydroxyethylacrylate/sodium acryloyldimethyl taurate copolymer with squalane and polysorbate 60, and SIMULGEL EPG™ which comprises a blend of a sodium acrylate/sodium acryloyldimethyltaurate copolymer with polyisobutene and caprylyl capryl glucoside.
Acrylate homopolymers, Acrylate crosspolymers, such as, but not limited to, Acrylate/C10-30 Alkyl Acrylate crosspolymers, carbomers, such as, but not limited to, acrylic acid cross linked with one or more allyl ethers, such as, but not limited to, allyl ethers of pentaerythritol, allyl ethers of sucrose, allyl ethers of propylene, and combinations thereof as are available as the Carbopol® 900 series from Noveon, Inc. of Cleveland, Ohio (e.g., Carbopol® 954).
Naturally occurring polymers such as xanthan gum, Galactoarabinan and other polysaccharides.
Combinations of the above rheology modifiers.

Examples of commercially available rheology modifiers include, but are not limited to: Ultrez™-10, a carbomer, and Pemulen™ TR-2, an Acrylate crosspolymer, both of which are available from Noveon of Cleveland, Ohio, and Keltrol™, a xanthan gum, available from CP Kelco of San Diego, Calif.

Rheology modifiers, when present, may be used in the present invention at a weight of the rheology modifier/weight of the total composition % (w/w) from about 0.01% to about 3%, in another embodiment from about 0.015% to about 2%, and in yet another embodiment from about 0.02% to about 1%.

Optional Components of the Cleansing Composition

Preservative:

The need to control microbiological growth in personal care products may be known to be particularly necessary in water based products.

The cleansing composition of the present invention may comprise a preservative compound or a combination of preservative compounds, of the same or different class, acting together as a preservative system. Preservative and preservative systems are used interchangeably in the present document to indicate one unique or a multiplicity of preservative compounds.

Preservatives prevent the growth of micro-organisms in the liquid lotion and/or on the substrate. Generally, such preservatives are hydrophobic or hydrophilic organic molecules. Suitable preservatives include, but are not limited to parabens, such as methyl paraben, ethyl paraben, propyl paraben, butyl paraben, and isobutyl paraben, alkyl glycinates, iodine derivatives, quaternary ammonium salts (e.g., benzalkonium chloride), and combinations thereof. Some preservative systems are disclosed in published U.S. Pat. Application Ser. No. 2004/022158 and in U.S. Pat. Application Ser. No. 10/878,875.

Adjunct Ingredient

The cleansing composition of the present invention may optionally include an adjunct ingredient. The adjunct ingredient may include a wide range of additional ingredients such as, but not limited to, perfumes, fragrances, humectants, texturizers, colorants, vitamins, botanical extracts, antioxidants and medically active ingredients, in particular, healing actives and skin protectants. Combinations of adjunct ingredients are also within the scope of the present invention.

Humectants are hygroscopic materials that function to draw water into the stratum corneum to hydrate the skin. The water may come from the dermis or from the atmosphere. Examples of humectants include, but are not limited to, glycerin, propylene glycol, and phospholipids. Fragrance components, such as perfumes, include, but are not limited to water insoluble oils, including essential oils. Vitamins include, but are not limited to, tocopherol acetate (Vitamin E acetate) and panthenol.

Botanical extracts are extracts containing the chemically active components of various plants and plant substances. Extracted botanical actives can include any water-soluble or oil-soluble active extracted from a particular plant. In addition, the botanical extracted actives can be supplied as a powder. Examples of suitable extracted botanical actives may be, but are not limited to, actives extracted from Echinacea (Echinacea angustifolia, Echinacea purpurae, Echinacea pallida), yucca glauca, willow herb, basil leaves, Turkish oregano, carrot root, grapefruit fruit, fennel fruit, rosemary, thyme, blueberry, bell pepper, black tea (Flowery Orange Pekoe, Golden Flowery Orange Pekoe, Fine Tippy Golden Flowery Orange Pekoe), blackberry, black currant fruit, Chinese tea, Japanese tea, green Darjeeling tea, coffee seed, dandelion root, date palm fruit, ginkgo leaf, green tea polyphenols (i.e., including epicatechin gallate and epigallocatechin 3-O -gallate), hawthorn berries, licorice, oolong tea, sage, strawberry, sweet pea, tomato, vanilla fruit, neohesperidin, quercetin, rutin, morin, myricetin, chlorogenic acid, glutathione, glycyrrhizin, absinthe, arnica, Centalla asiatica, chamomile, comfrey, cornflower, horse chestnut, ivy (Herdera helix), magnolia, mimosa, oat extract, pansy, scullcap, seabuckthorn, white nettle, witch hazel, aloe, sunflower, lavender, vanilla, milk, butter milk, avocado, jojoba, coconut, green tea, calendula, sweet almond, and any combinations thereof.

Preparation of the Cleansing Composition

The process of the invention can be seen as a three step process, where a concentrated emulsion composition may be prepared first. The concentrated composition can later be diluted and complemented with other compounds. The diluted composition can then be applied to wipes substrate material.

In general, traditional lotion compositions for wet-wipes, and more specifically emulsions are manufactured by mixing an emollient compound into an excess of water and a surfactant or emulsifier. This process for making an emulsion, however, requires stringent process conditions, i.e., a high energy input to homogenize the oil and water phase and to generate a defined particle size distribution in the aqueous phase. Generally, high shear mixing may be needed together with heating of the composition being prepared.

This high-energy input may be costly for the manufacturer and may require specific equipment (which may be a significant investment). Also, the high shear and stringent process conditions represent a significant stress on the ingredients of emulsions conventionally prepared. For example, break-down of large molecular weight structures may occur. Moreover, the emulsions prepared by such conventional processes can be by nature very diluted. Handling, storage and transportation represent significant inconvenience and cost.

Also, there may be a limit in emollient droplet size which can be achieved with traditional emulsion making processes. The droplet size is the size of the oil droplets in the aqueous solution of the oil-in-water emulsion. Emulsions of small oil droplet size generally require more stringent preparation whereas it has been shown that small oil droplet size induces more desirable end properties for the emulsions. While not intending to be bound by theory, it has been found that emulsions with small oil droplet size deliver to the user an increased amount of the emollient (and anything co-emulsified with the emollient) upon application.

The present invention provides a process for making a wet wipe comprising a cleansing composition. The present invention provides a process for preparing the cleansing composition as a concentrated emulsion composition that can be easily handled, stored and transported. The concentrated emulsion composition can then be diluted with water and complemented with other compounds. The result may be an oil-in-water emulsion that exhibits small oil droplet size, when diluted. The desired final cleansing composition may then be applied to a wipe substrate. In that respect, the present invention allows to best leverage manufacturing capability by providing a concentrated emulsion composition that can be diluted into a ready-to-use cleansing composition.

The process may be characterized by the fact that it requires only a low level of energy input (e.g., low shear and little or no heat input) to ensure the formation of a stable and/or meta-stable emulsion composition concentrate, compared to conventional processes.

In one process, a powder skin health benefit ingredient may be added to an emollient, or mixture of emollients, and mixed until the powder skin health benefit ingredient is uniformly distributed. The emollient/powder skin health ingredient may be added to an emulsifier, or mixture of emulsifiers, forming a concentrated mixture. The concentrate may then be diluted with water and may also be complemented with other compounds.

In a second process, a pre-dispersion of the skin-health benefit ingredient in an emollient, or mixture of emollients, and optionally including a dispersing agent may be added to an emulsifier, or mixture of emulsifiers, to form the concentrated mixture. The concentrate may then be diluted with water and may also be complemented with other compounds.

Shear rate:

Contrary to the conventional emulsification process using very high shear rate, the present invention allows for using a shear rate below about 10,000s⁻¹, in another embodiment below 1,000s⁻¹, and in yet another embodiment below 100s⁻¹.

Converting:

The process of the present invention comprises a step of providing a wipe substrate and providing a quantity of the cleansing composition onto the substrate. This step is referred to as a converting step. Any known type of substrate may be used in the converting step. In one embodiment the substrate may be of a synthetic nonwoven nature. However, woven materials, as well as the use of natural compounds in either woven or nonwoven materials are within the scope of the present invention. The step of providing the emulsion composition onto the wipes substrate may be achieved by any conventional application process, such as, but not limited to, spraying, printing, and coating, for example, with the use of a curtain coater or slot coater.

### EXAMPLES

Examples A, B, C, D, and E are examples of the present invention in which the skin health benefit ingredient is incorporated into the oil phase of the composition.

| Component | Amount (% by weight) |
|---|---|
| Decyl glucoside/water 50/50 | 0.10 |
| Abil Care 85 ™* | 0.90 |
| Arlatone V-175 ™** | 0.50 |
| Propylene Glycol | 1.50 |
| Methyl Paraben | 0.15 |
| Ethyl Paraben | 0.05 |
| Propyl Paraben | 0.05 |
| Benzyl Alcohol | 0.50 |
| ZNO-350 ™ ZnO powder | 1.00 |
| Purified Water | 95.25 |
| Total | 100.00 |

*Abil Care 85 ™ comprises Caprylic Capric Triglyceride and Bis-PEG/PPG-16/16 PEG/PPG-16/16 Dimethicone as is available from Degussa Care Specialties in Hopewell, VA.
**Arlatone V-175 ™ comprises sucrose palmitate, glyceryl stearate, glyceryl stearate citrate, sucrose, mannan, and xanthan gum as is available from Uniqema GmbH&Co. KG in Emmerich, Germany.

| Component | Amount (% by weight) |
|---|---|
| Decyl glucoside/water 50/50 | 0.40 |
| Polysorbate-20 | 0.08 |
| Xanthan Gum | 0.30 |
| Propylene Glycol | 1.50 |
| Methyl Paraben | 0.15 |
| Ethyl Paraben | 0.05 |
| Propyl Paraben | 0.05 |
| Sodium Benzoate | 0.20 |
| Spectraveil MOTG ™ ZnO dispersion | 3.60 |
| Purified Water | 93.67 |
| Total | 100.00 |

| Component | Amount (% by weight) |
|---|---|
| Decyl glucoside/water 50/50 | 0.20 |
| Arlatone V-175 ™ | 0.50 |
| Propylene Glycol | 1.50 |
| Methyl Paraben | 0.15 |
| Ethyl Paraben | 0.05 |
| Propyl Paraben | 0.05 |
| Benzyl Alcohol | 0.50 |
| ZNO-350 ZnO powder dispersed in Abil | 1.80 |
| Care 85 ™ | |
| Purified Water | 95.25 |
| Total | 100.00 |

| Component | Amount (% by weight) |
|---|---|
| Decyl glucoside/water 50/50 | 0.10 |
| Abil Care 85 ™ | 0.90 |
| Arlatone V-175 ™ | 0.50 |
| Propylene Glycol | 1.50 |
| Methyl Paraben | 0.15 |
| Ethyl Paraben | 0.05 |
| Propyl Paraben | 0.05 |
| Benzyl Alcohol | 0.50 |
| Zinc Carbonate | 1.00 |
| Purified Water | 95.25 |
| Total | 100.00 |

| Component | Amount (% by weight) |
|---|---|
| Decyl glucoside/water 50/50 | 0.60 |
| Polysorbate-20 | 0.08 |
| Abil Care 85 ™ | 1.80 |
| Myritol 318 ™ | 1.80 |
| Xanthan Gum | 0.20 |
| Propylene Glycol | 1.50 |
| Methyl Paraben | 0.15 |
| Ethyl Paraben | 0.05 |
| Propyl Paraben | 0.05 |
| Benzyl Alcohol | 0.50 |
| Zinc Stearate | 1.80 |
| Purified Water | 91.47 |
| Total | 100.00 |

### TEST METHODS

Lability Method

Particulate zinc materials differ with respect to how strongly the zinc ion (Zn²⁺) is held by the counterion or counterions. The skin health benefits discussed herein depend upon having available Zn²⁺. To determine which particulate zinc materials provide labile Zn²⁺, a test is developed using a metallochromic dye that changes color upon coordinating Zn 2+. The response is a binary visual assessment of whether or not the color changes, indicating zinc-binding. If the color changes, the particulate zinc material is classified as having available Zn²⁺.

The method is based on the commercial metallochromic dye Zincon sodium salt (2-carboxy-2′-hydroxy-5′-sulfoformazyl-benzene sodium salt, such as Cat. No. 201332 available from Sigma-Aldrich, St. Louis, Mo.). Zincon changes from an orange to blue color upon binding zinc ion and provides the basis for detecting available Zn 2+. The specific procedure involves making a stock solution of Zincon in ethanol (50 mg/10 ml ethanol). The particulate zinc material is then added to water (30 mg/10 ml water) and agitated vigorously (pH should be 7-9). Three to four drops of Zincon solution are then added to the particulate zinc material in water, agitated, and a visual assessment of color change made.

Epiderm Culture Assay With Quantification of IL-1α and Viability

In order to evaluate the efficacy of the materials and embodiments of the present invention, a bio-engineered human skin construct is used to model the response of normal human epidermis. The EPIDERM™ skin model (as available from MatTek Corporation, Ashland, Mass.) is a three-dimensional, cornified, air-interfaced culture system. Each EPIDERM™ culture has multiple layers of progressively differentiated keratinocytes, closely resembling human epidermis. EPIDERM™ EPI-200-hydrocortisone-free (hereinafter EPI-200-HCF) cultures are used because these cultures show a strong inflammatory response to irritants. Cultures are incubated in medium that is also hydrocortisone-free (EPI-100-MM-HCF). All work with the cultures is carried out under sterile conditions in a biological safety cabinet.

Experiments using the EPI-200-HCF cultures are conducted in six-well plates supplied with the cultures. Each well of the plate is filled with 900 microliters of pre-warmed EPI-100-MM-HCF medium, and then one EPI-200-HCF culture insert (shipped in 24-well format) is carefully transferred into the center of each well such that no air bubbles are trapped underneath the insert. After all of the culture inserts are transferred, the six-well plates are incubated in a 37° C., 5% CO₂ tissue culture incubator for at least two hours.

During the time the cultures are acclimating, the controls and test samples to be applied to the cultures are prepared. Sufficient material is prepared such that quadruplicate cultures can be dosed with about 100 microliters of the control or test sample. Throughout this section, the word “water” refers to autoclaved, Milli-Q water.

For each experiment, three controls are run. The first control is water. The second control is a freshly-made solution of 0.125% Triton-X-100 in water, prepared by diluting the 1% Triton-X-100 stock solution shipped with the EPIDERM™ cultures. The third control is infant irritating bowel movement (hereinafter IIBM). IIBM is prepared as described in the PREPARATION OF INFANT IRRITATING BM method of the TEST METHODS section. At the time of use, the required number of tubes of IIBM are thawed at room temperature and 250 microliter aliquots are dispensed into 1.5 ml polypropylene microfuge tubes using a positive displacement pipettor. To each microfuge tube is then added 10 microliters of a 1 N NaOH solution to raise the pH and ensure activity of the fecal enzymes. For the IIBM control, an additional 250 microliters of water is added, and the tube is mixed thoroughly using a vortex mixer.

For preparation of the test samples, a suspension in water of the material to be tested is first prepared. As an example, a 1% suspension of zinc oxide in water is prepared by weighing out 0.1 grams of zinc oxide into a 15 ml polypropylene tube (such as Cat No. 21008-935 from VWR) and adding sufficient water to bring the volume to 10 milliliters. Each suspension is vortexed thoroughly and allowed to sit for at least one hour at room temperature prior to use. To ascertain the irritancy of the test sample by itself, the neat suspension is applied to the EPIDERM™ culture. To ascertain the ability of the test sample to reduce the irritancy of IIBM, 250 microliters of the suspension is added to a microfuge tube already containing a 250 microliter aliquot of IIBM plus 10 microliters of 1 N NaOH (as prepared above). The BM-test sample mixture is vortexed thoroughly and allowed to sit for one hour prior to application to the EPIDERM™ culture. Note that the concentration of the test sample, when mixed with IIBM, will be halved. Consequently, a 2× suspension should be prepared when mixing with IIBM is desired. All suspensions are vortexed thoroughly immediately before adding to the EPIDERM™ culture or adding to IIBM. There should be no obvious settling of the suspension prior to transfer. A positive displacement pipettor should be used to transfer the suspension.

After the controls and test samples are prepared and the EPIDERM™ cultures are acclimated, the cultures are removed from the incubator and the underlying medium is removed by aspiration and replaced with 900 microliters of fresh, pre-warmed EPI-100-MM-HCF medium. Care must be taken to ensure that no air bubbles are captured underneath the culture inserts. Any residual medium on the surface of the EPIDERM™ cultures is gently aspirated off, and about 100 microliters of a control or test sample (neat or mixed with IBM) is applied to quadruplicate cultures using a positive displacement pipettor. The dosed cultures are then incubated at 37° C., 5% CO₂ for 16 hours.

At the end of the incubation, the plates are removed from the incubator and the underlying medium is collected and stored, preferably in a 96-well deep well plate (such as Cat No. BK267006 from VWR). Internal studies have shown that, for analysis of IL-1α, the medium can be stored overnight in a 4° C. refrigerator. If the medium is not to be analyzed in a 24 hour time period, it is frozen and stored at −80° C.

The inflammatory response of the EPIDERM™ culture to the controls or test samples is quantified by measuring the production of IL-1α using a Human IL-1α Quantikine ELISA Kit (Cat. No. DLA50, R&D Systems, Minneapolis, Minn.) according to instructions provided with the kit, except that 40 microliters of collected medium are added to 160 microliters of the RD5 calibrator diluent in step 4 of the instruction manual, i.e. a 5× dilution of the collected medium (in calibrator diluent RD5) is analyzed. The average and standard error of the mean (SEM) is calculated for each set of quadruplicate cultures. If necessary, outlier analysis may be used to identify and remove data associated with erroneous EPIDERM™ cultures. Such analysis is described on page 460 of the book “Statistical Methods in Research and Production” edited by Owen L. Davies and Peter L. Goldsmith, published by Longman Group Limited, fourth revised edition (published in 1984).

In addition to measuring the amount of IL-1α produced, the viability of each EPIDERM™ culture at the end of the incubation period is also measured using a standard MTT (methylthiazoletetrazolium) assay. The reduction of the dye as a result of cellular metabolism can be used to measure the cytotoxicity of the treatment. Each culture insert is rinsed at least 3 times with phosphate buffered saline (shipped with the EPIDERM™ cultures) until the surface of the culture is visibly free of debris. Residual phosphate buffered saline on the cultures is gently aspirated off. Each culture insert is then placed into the well of a 24-well plate (provided with the cultures), into which has already been added 300 microliters of a 1 mg/ml MTT solution prepared in EPI-100-MM-HCF medium. Suitable MTT is available from Sigma-Aldrich of St. Louis, Mo. as Cat. No. M2128. Care must be taken not to trap air bubbles underneath the inserts. The cultures are incubated at 37° C., 5% CO₂ for three hours. After incubation, excess MTT solution is removed from the sides of each insert and each insert is transferred to a fresh 24-well plate and immersed in 2 milliliters of 100% isopropanol. Each 24-well plate is covered and placed into a ZIPLOCK™ bag to reduce evaporation. All plates are then allowed to sit overnight in the dark. Prior to removing extraction solution for analysis, each plate is placed on a titer plate shaker (such as Cat. No. 57019-600 from VWR) and gently swirled (˜100 rpm) for five minutes. After mixing is complete, 200 microliters of the extraction solution is removed from each well and transferred to a 96-well plate. The optical density of the samples is measured at 570 nm using a microplate reader (such as the SpectraMax M2 available from Molecular Devices Corporation, Sunnyvale, Calif.). A background reading at 650 nm is subtracted to improve data quality. Percent viability is normalized to the readings obtained for the water control. Treatments reducing the viability of the EPIDERM™ cultures below 70% have a propensity to be irritating when repeatedly dosed on real skin, especially under occluded conditions.

The percent mean reduction of IL-1α is calculated as follows:

$$\% mean reduction of IL \text{-} 1 α = 100 \times \frac{BM Control Result - Test Sample Result}{BM Control Result}$$

$$BM Control Result = mean measured amount of IL \text{-} 1 α from IIBM alone control$$

$$Test Sample Result = mean measured amount of IL \text{-} 1 α from IIBM \text{-} test sample treatment$$

The greater the percent mean reduction of IL-1α, the more effective the given treatment is at reducing irritation caused by exposure to IIBM.

Effectiveness, however, should not be at the expense of gentleness to skin, which is why viability of the EPIDERM™ cultures must also be quantified. The higher the viability of the cultures post-treatment, the less irritating the treatment. A discussion of viability and irritancy can be found in “Comparison of in Vitro and in Vivo Human Skin Responses to Consumer Products and Ingredients with a Range of Irritancy Potential” by M. Perkins et al., published in Toxicological Sciences 48:218-229 (1999). Particulate zinc materials of the present invention have surprisingly been found to decease viability of the EPIDERM™ cultures less than soluble zinc salts, when compared at an equimolar level of zinc. Consequently, particulate zinc materials are less irritating to skin than soluble zinc salts.

It is important to realize that when a given treatment results in low viability of the EPIDERM™ cultures (a normalized viability of less than 30%), it is not possible to accurately interpret the IL-1α data for that treatment. Dead keratinocytes do not produce IL-1α . Hence, the IL-1α data for a low viability treatment may be abnormally low and may make a treatment look effective while all it actually did was kill the culture.

The table below compares the percent mean reduction in IL-1α and viability data for zinc oxide, a particulate zinc material, and zinc sulfate, an aqueous zinc salt.

| | |
|---|---|
| Zinc Oxide | |
| Concentration (mM) | EPIDERM Zinc Oxide-IIBM Data |

| w/w % Shown | Mean % IL-1α | Normalized |
|---|---|---|
| in Parentheses | Reduction | EPIDERM Viability |
| 125 (1.0%) | 90 | 96 |
| 63 (0.5%) | 76 | 95 |
| 31 (0.25%) | 68 | 95 |

| | |
|---|---|
| Zinc Sulfate | |
| Concentration (mM) | EPIDERM Zinc Sulfate-IIBM Data |

| w/w % Shown | Mean % IL-1α | Normalized |
|---|---|---|
| in Parentheses | Reduction | EPIDERM Viability |
| 174 (5.0%) | 98 | 14 |
| 174 (5.0%) | 99 | 11 |
| 87 (2.5%) | 99 | 44 |
| 87 (2.5%) | 98 | 28 |

It is readily apparent that treatment with the aqueous zinc salt results in lower viability of the EPIDERM cultures and, therefore, has greater potential to irritate real skin. A unique finding of the present invention is that particulate zinc material, with a smaller impact on viability, is more gentle while still being effective at reducing irritation caused by IIBM. As such, particulate zinc material may be better suited for use on delicate baby skin, especially when such skin is damaged by diaper rash.

Preparation of Infant Irritating BM (IIBM)

Individual samples of infant BM are collected from soiled diapers, and each sample is characterized in terms of how much IL-1α production it elicits when added to a monolayer culture of pooled neonatal human epidermal keratinocytes (available from Cambrex Bio Science, Baltimore, Md. as Cat. No. CC-2507). The keratinocytes are plated and grown in KGM-2 (Cambrex Bio Science, Cat. No. CC-3107) according to instructions provided by the manufacturer. After incubating the monolayer with a BM sample for 16 hours, the medium is collected, briefly spun to remove particulates, and the IL-1α content is quantified using a Human IL-1α Quantikine ELISA Kit (Cat. No. DLA50, R&D Systems, Minneapolis, Minn.). A pooled IIBM sample previously prepared and verified to be irritating when applied to EPIDERM cultures (using the EPIDERM culture assay described earlier) is included as a control. New BM samples that elicit a response similar to or greater than that of the control are each mixed 1:1 with water, blended with a stomacher (such as the Stomacher 400C available from Brinkmann Instruments, Westbury, NY, as Cat. No. 030010108), and mixed together to form a pool. This pool is dispensed into 15 ml polypropylene tubes and frozen at −80° C. until use.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cleansing composition comprising:
a. an oil phase comprising
i. an emollient,
ii. an emulsifier,
iii. a particulate skin health benefit ingredient;
b. a water phase comprising
i. a rheology modifier.

2. The cleansing composition of claim 1 wherein said cleansing composition has a pH from about 6.0 to about 9.0.

3. The cleansing composition of claim 1 wherein said cleansing composition reduces the IL-1α response to irritating infant BM by at least about 40% while maintaining a cell viability of greater than about 70%.

4. The cleansing composition of claim 1 wherein said particulate skin health benefit ingredient is selected from the group consisting of zinc aluminate, zinc carbonate, zinc oxide, zinc phosphate, zinc selenide, zinc sulfide, zinc silicate, zinc silicofluoride, zinc borate, zinc hydroxide, zinc hydroxy sulfate, hydrozincite, basic zinc carbonate, aurichalcite, rosasite, zinc hydroxychloride, zinc hydroxynitrate, sphalerite, wurtzite, smithsonite, franklinite, zincite, willemite, troostite, hemimorphite, zinc caproate, zinc laurate, zinc oleate, zinc stearate, zinc undecylate, zinc alkyl sulfonic acids, zinc naphthenate, zinc tartrate, zinc tannate, zinc monoglycerolate, zinc allantoinate, zinc urate, zinc amino acids, zinc polycarboxylates, zinc polysulfate, zinc arginine, zinc histidine, zinc hexaborate, zinc oxalate, gold containing material, silver containing material, platinum containing material and combinations thereof.

5. The cleansing composition of claim 1 wherein said emollient has a solubility parameter between about 5 and about 12.

6. The cleansing composition of claim 1 wherein said emollient has a solubility parameter between about 5 and about 9.

7. The cleansing composition of claim 1 wherein said emollient is selected from the group consisting of Cyclomethicone, Dimethiconol, Dimethicone, Cyclopentasiloxane, Caprylic/Capric Triglyceride, C₁₂-C₁₅ Alkylbenzoate, Bis-PEG/PPG-16/16 PEG/PPG-16/16 Dimethicone, mineral oil, phenyl-modified silicones, alkyl-modified silicones, silicone resins and combinations thereof.

8. The cleansing composition of claim 1 wherein said emulsifier is selected from the group consisting of alkylpolyglucosides, decylpolyglucosides, fatty alcohols, poly-12 Hydroxy Stearic Acid, Caprylic Capric Triglyceride, Bis-PEG/PPG-16/16 PEG/PPG-16/16 Dimethicone, Polysorbate 20, and combinations thereof.

9. The cleansing composition of claim 1 wherein said cleansing composition further comprises a preservative.

10. The cleansing composition of claim 9 wherein said preservative is selected from the group consisting of methyl paraben, ethyl paraben, propyl paraben, butyl paraben, isobutyl paraben, alkyl glycinates, iodine derivatives, quaternary ammonium salts, and combinations thereof.

11. The cleansing composition of claim 1 wherein said rheology modifier is selected from the group consisting of blends of sucrose palmitate, glyceryl stearate, glyceryl stearate citrate, sucrose, mannan, xanthan gum; blends of Steareth-100, Steareth-2, glyceryl stearate citrate, sucrose, mannan, xanthan gum; blends of hydroxyethylacrylate/sodium acryloyldimethyltaurate copolymer, polyisobutene, caprylyl capryl glucoside; Acrylate homopolymers; Acrylate crosspolymers; xanthan gum; Galactoarabinan, and combinations thereof.

12. A wet-wipe comprising a non-woven material; said non-woven material releasably carrying said cleansing composition of claim 1.

13. The wet-wipe of claim 12 wherein said non-woven material comprises fibers comprising a material selected from the group consisting of polyolefin, polyester, cellulose, rayon, lyocell, polyamide, polyesteramide, polyvinyl alcohol, and combinations thereof.

14. A wet-wipe comprising a non-woven material; said non-woven material releasably carrying said cleansing composition of claim 1 wherein said particulate skin health benefit ingredient has a solubility of less than about 1% in water at 25° C.

15. A wet-wipe comprising a non-woven material; said non-woven material releasably carrying said cleansing composition of claim 1 wherein said particulate skin health benefit ingredient, when tested as a 2% suspension in water, does not reduce skin cell viability below 70% according to the EPIDERM™ test method.

16. A process for making a wet-wipe comprising the steps of:
a. Preparing a concentrated emulsion composition; said step of preparing a concentrated emulsion composition comprising the steps of:
i. Combining an emollient, an emulsifier, and a particulate skin health benefit ingredient;
ii. Mixing said combination to obtain a substantially homogeneous concentrated emulsion composition;
b. Preparing a diluted composition by diluting said concentrated emulsion composition with water; and
c. Providing a wipe substrate and providing a quantity of said diluted composition onto said wipe substrate.

17. The process for making a wet-wipe of claim 16 wherein said skin health benefit ingredient is pre-dispersed in at least one emollient.

18. The process of claim 16 wherein said concentrated emulsion composition has an average emollient droplet size of less than about 12 microns, when diluted in an excess of water.

19. The process of claim 16 wherein step l(a)(ii) is performed at a shear rate of less than about 10,000s⁻¹.

20. The process of claim 16 wherein said concentrated emulsion composition has a water content level of from about 0% to about 30%.

21. The process of claim 16 wherein said concentrated emulsion composition has a water content level of from about 0% to about 20%.

22. The process of claim 16 wherein said concentrated emulsion composition has a water content level of from about 0% to about 10%.

23. The process of claim 16 further comprising a step (d) of adding an adjunct ingredient into said concentrated emulsion composition or into said diluted composition prior to step (c).

24. The process of claim 23 wherein said adjunct ingredient is selected from the group consisting of soothing agents, vitamins, minerals, botanicals, perfumes, antioxidants, potentiators, aesthetic enhancing ingredients, preservatives, fragrances, humectants, texturizers, colorants, medically active ingredients, healing actives, skin protectants and combinations thereof.

25. A wet-wipe prepared according to the process of claim 16.

26. A wet-wipe prepared according to the process of claim 17.

27. A plurality of wet-wipes prepared according to the process of claim 16.

28. A plurality of wet-wipes prepared according to the process of claim 17.
